(19) United States
(12) Patent Application Publication
(10) Pub. No.: US 2026/0144565 A1
(43) Pub. Date: May 28, 2026

(54) CLOT VISUALIZATION AND WORKFLOWS UTILIZING REAL-TIME IMAGING FOR THROMBUS REMOVAL SYSTEMS AND METHODS

(71) Applicant: Shifamed Holdings, LLC, Campbell, CA (US)

(72) Inventors: Muralidharan SRIVATHSA, Santa Clara, CA (US); Aadel AL-JADDA, San Carlos, CA (US); Praveen Krishna DALA, San Ramon, CA (US)

(21) Appl. No.: 19/123,721

(22) PCT Filed: Oct. 31, 2023
(86) PCT No.: PCT/US2023/078286

§ 371 Date: Apr. 23, 2025

Related U.S. Application Data
(60) Provisional application No. 63/381,615, filed on Oct. 31, 2022.

(51) Int. Cl. A61B 17/3203; A61B 17/00; G06T 7/00; G06T 7/62

(52) U.S. Cl. CPC A61B 17/32037; G06T 7/0012; G06T 7/62; A61B 2017/00057; A61B 2017/00128; A61B 2217/005; G06T 2207/10081; G06T 2207/30104

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application no. 63/381,615, titled “CLOT VISUALIZATION AND WORKFLOWS UTILIZING REAL-TIME IMAGING FOR THROMBUS REMOVAL SYSTEMS AND METHOD”, and filed on Oct. 31, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days. More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4D illustrate various embodiments of a thrombus removal system including a saline source, an aspiration system, and one or more controls for controlling irrigation and/or aspiration of the system.

FIGS. 5A-5D illustrate embodiments of workflows and features of the thrombus removal system for imaging clots and estimating the sizes of clots.

FIG. 6 is a flowchart of a thrombectomy method.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A method of estimating progress of a thrombectomy procedure is provided, comprising: performing pre-operative medical imaging of one or more clots in a patient; determining a pre-operative parameter of the one or more clots based on the pre-operative medical imaging; removing at least a portion of the one or more clots with a thrombectomy device; determining a parameter of the removed portion of the one or more clots; and displaying the parameter of the removed portion of the one or more clots to a user.

In some aspects, the pre-operative medical imaging comprises CT imaging.

In one aspect, the pre-operative parameter comprises a volume of the one or more clots.

In other aspects, the pre-operative parameter comprises one or more dimensions of the one or more clots.

In one aspect, the pre-operative parameter comprises a weight of the one or more clots.

In some aspects, the pre-operative parameter comprises a morphology of the one or more clots.

In one aspect, the parameter of the removed portion comprises a volume of the one or more clots removed.

In other aspects, the parameter of the removed portion comprises one or more dimensions of the one or more clots removed.

In other aspects, the parameter of the removed portion comprises a weight of the one or more clots removed.

In some aspects, the parameter of the removed portion comprises a morphology of the one or more clots removed.

In one aspect, determining the pre-operative parameter comprises performing image processing to estimate the parameter of the one or more clots.

In other aspects, determining the parameter of the removed portion comprises sensing a weight of the one or more clots after removal.

In some aspects, determining further comprises weighing the one or more clots in a clot capture container.

In one aspect, determining the parameter of the removed portion comprises evaluating one or more images of the one or more clots after removal.

In other aspects, the method comprises comparing the pre-operative parameter to the parameter of the removed portion of the one or more clots.

In another aspect, the method comprises displaying a percentage of the one or more clots removed.

In some aspects, the method includes overlaying a graphic of portions of the one or more clots removed on top of a graphic of the pre-operative medical imaging of the one or more clots.

In one aspect, the method comprises filtering blood and/or saline from the one or more removed clots.

In another aspect, the method includes comparing a pre-operative weight of the one or more clots to a weight of the one or more clots removed.

In some aspects, the method comprises comparing a pre-operative dimension of the one or more clots to a dimension of the one or more clots removed.

In some aspects, the method includes comparing a pre-operative volume of the one or more clots to a volume of the one or more clots removed.

In some aspects, removing at least a portion of the one or more clots comprises: directing one or more fluid streams towards the one or more clots to break up the one or more clots; and aspirating the one or more clots with the thrombectomy device.

In some aspects, the method includes calculating a volume of saline delivered with the one or more fluid streams.

In another aspect, the method includes separating fluid from the removed portion of the one or more clots.

In some aspects, the method includes calculating a volume of blood removed from the patient by subtracting the volume of saline delivered from a volume of the separated fluid.

A thrombectomy system is provided, comprising: an elongate catheter having an aspiration lumen adapted to be inserted into a subject adjacent to one or more target clots; an aspiration source coupled to the aspiration lumen; a clot capture container fluidly coupled to the aspiration lumen; one or more sensors configured to measure or sense at least one parameter of removed clots captured in the clot capture container; and a display configured to indicate the at least one parameter to a user of the thrombectomy system.

In some aspects, one or more processors are operatively coupled to the one or more sensors and the display, the one or more processors being configured to evaluate pre-operative imaging of the one or more target clots of the subject to calculate a pre-operative parameter of the one or more target clots, the one or more processors being configured to compare the pre-operative parameter to the parameter from the one or more sensors.

In one aspect, the one or more processors being further configured to provide a comparison between the pre-operative parameter and the at least one parameter from the one or more sensors to be presented on the display.

In another aspect, the pre-operative parameter comprises a volume of the one or more clots.

In some aspects, the pre-operative parameter comprises one or more dimensions of the one or more clots.

In other aspects, the pre-operative parameter comprises a weight of the one or more clots.

In some aspects, the pre-operative parameter comprises a morphology of the one or more clots.

In another aspect, the parameter of the removed portion comprises a volume of the one or more clots removed.

In some aspects, the parameter of the removed portion comprises one or more dimensions of the one or more clots removed.

In one aspect, the parameter of the removed portion comprises a weight of the one or more clots removed.

In another aspect, the parameter of the removed portion comprises a morphology of the one or more clots removed.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

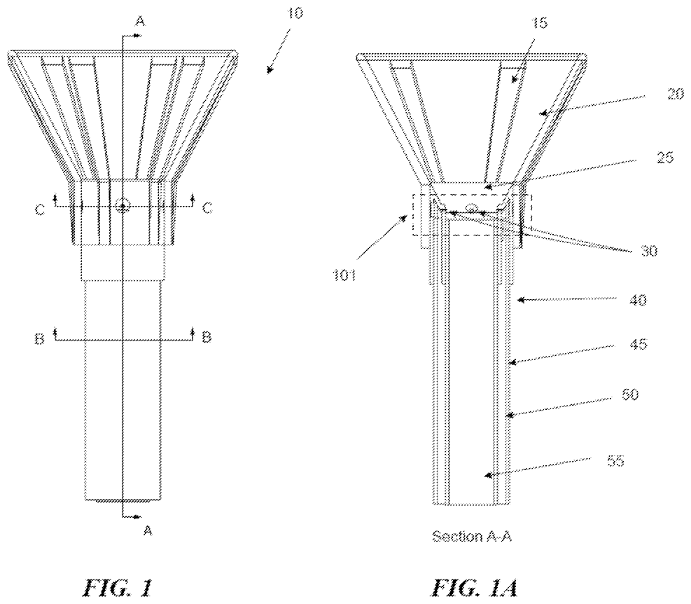

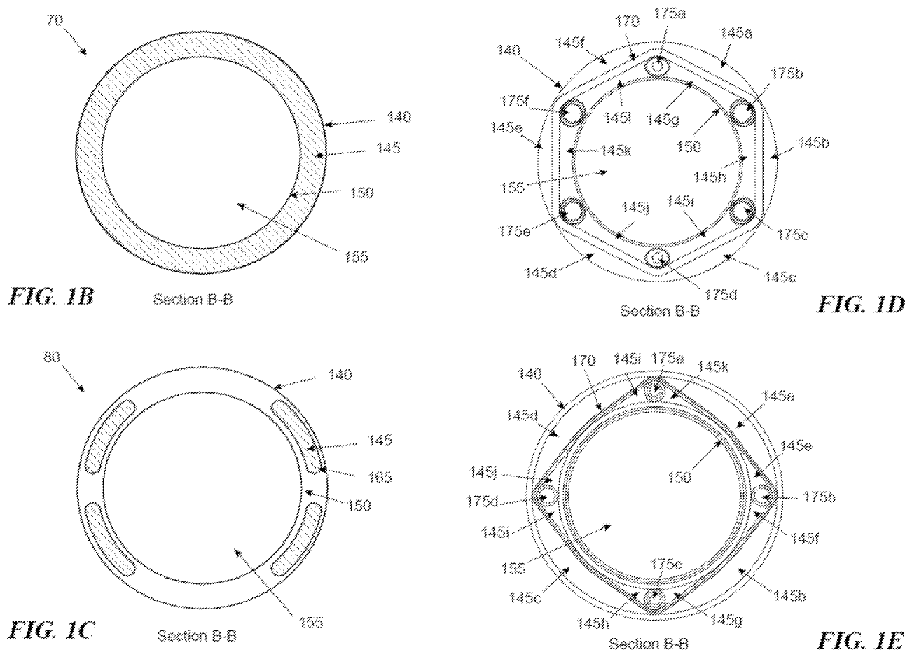

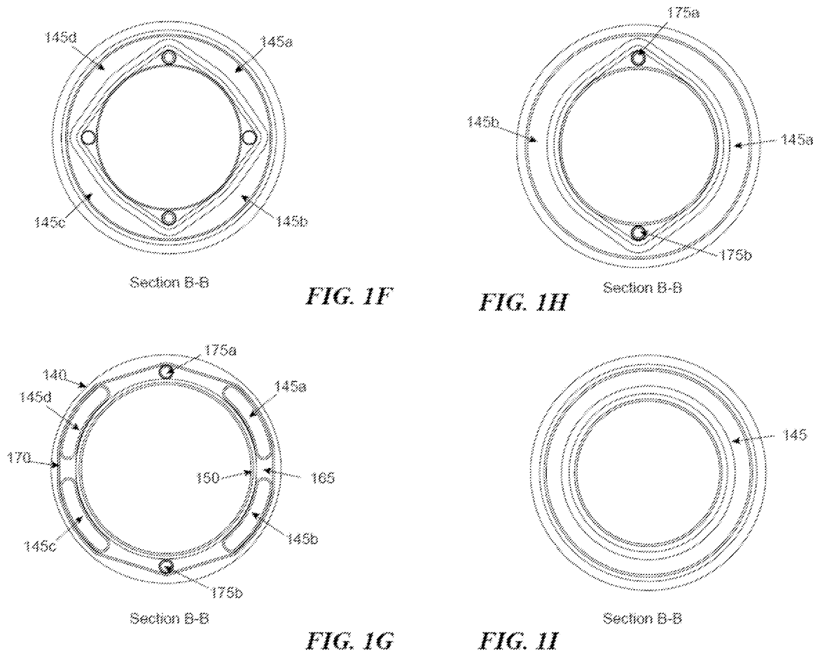

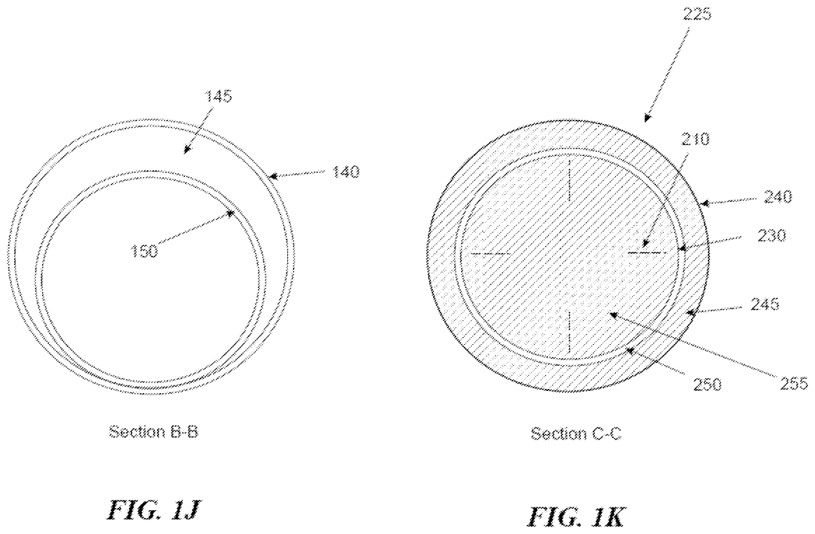

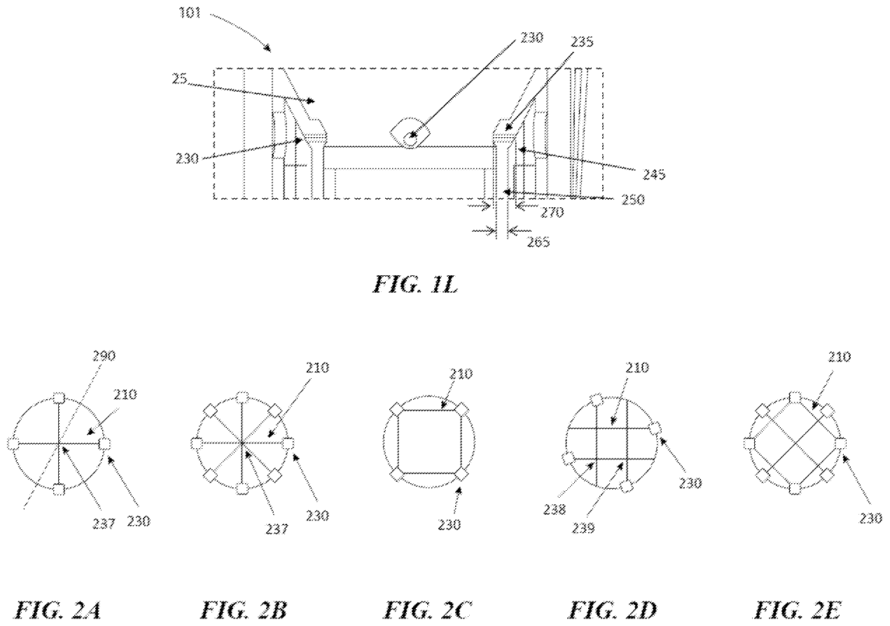

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-14l and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15m/s to up to 150 m/s. At these higher velocities (e.g. above 15m/s, or alternatively above 20m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

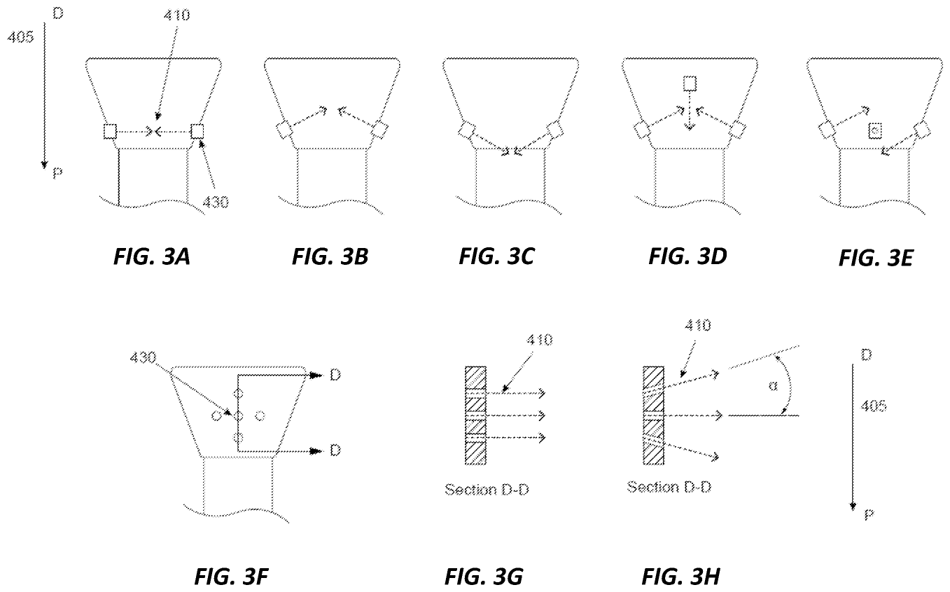

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

### Cavitation Generation

The exemplary system includes fluidic jets configured in a particular manner to enhance removal of clot. The exemplary fluid streams or jets have been shown in bench studies to dramatically improve removal of clot through various mechanisms of action optionally including, but not limited to, cavitation and water cutting. In contrast to conventional fluid mechanisms for thrombectomy, in some embodiments herein, fluid streams from respective ports are delivered at sufficient flow rates (and patterns) to create cavitation and/or other preferential effects to improve removal of clot. In certain examples, the cavitation effect is created by large pressure drops and deceleration at the focal point and/or intersection point of at least two fluid streams. The cavitation may provide a source of turbulent kinetic energy that can be used to mechanically fractionate and/or liquefy thrombi or other target tissue structures. When the fluid velocity is sufficiently high, the material accumulates impact energy, which can cause deformation and fragmentation. This also may modify the surface properties of the clot to allow the material to be penetrated to enable cavitation within the clot. Collision or interaction of the high-speed jets creates hydrodynamic cavitation whereby a pressure drop below the vapor pressure of the liquid creates bubbles which eventually collapse with great mechanical energy in the cavitation field, causing a kind of implosion in the clot material. Further, with multiple jets directed towards a focal point or sufficiently near respective streams, the closing speed of the fluid particles is significantly higher (up to double) that of a single jet stream. This also forces fluid and/or particles out from the space between the fluid jets at high speed. The speed of the fluid jets is sufficiently high to create a pressure drop below the vapor pressure such that the fluid vaporizes. When pressure rises again the bubble collapses, which causes the cavitation. It has been found that the power of the exemplary system and cavitation effect significantly exceeds conventional fluid jet(s) and mechanical tools like rotating screws. In some examples, the collapse of the bubbles may generate heat in or around the target tissue, which may further promote breaking up of the clot. In bench studies systems in accordance with various embodiments were able to remove certain clot material that simple aspiration or water jetting were not. In other studies, the exemplary systems were able to remove clot material in a fraction of the time of conventional systems.

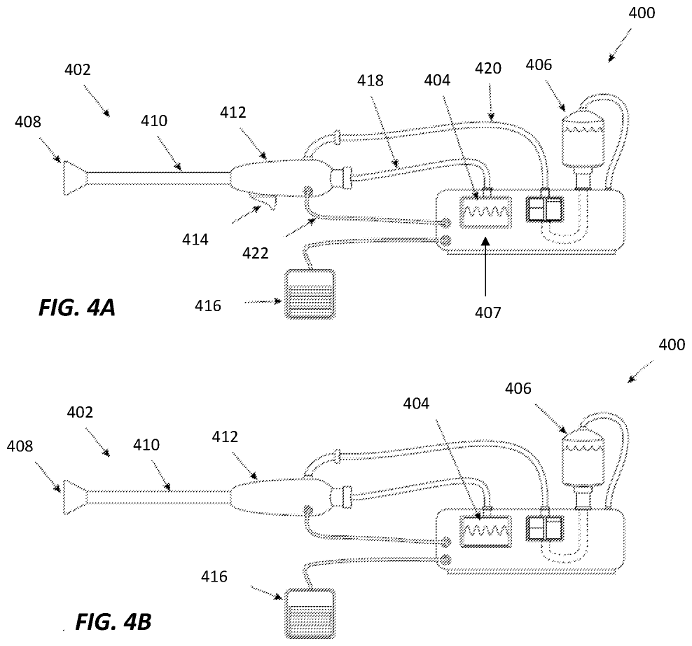

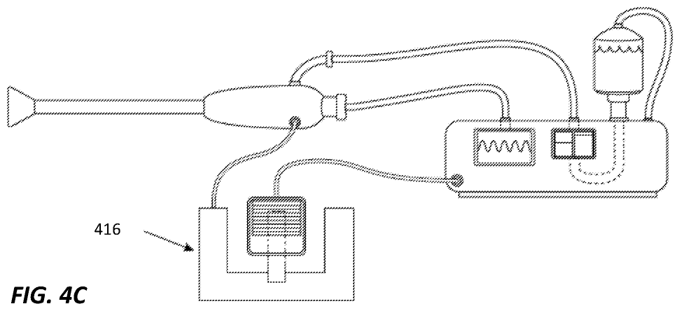

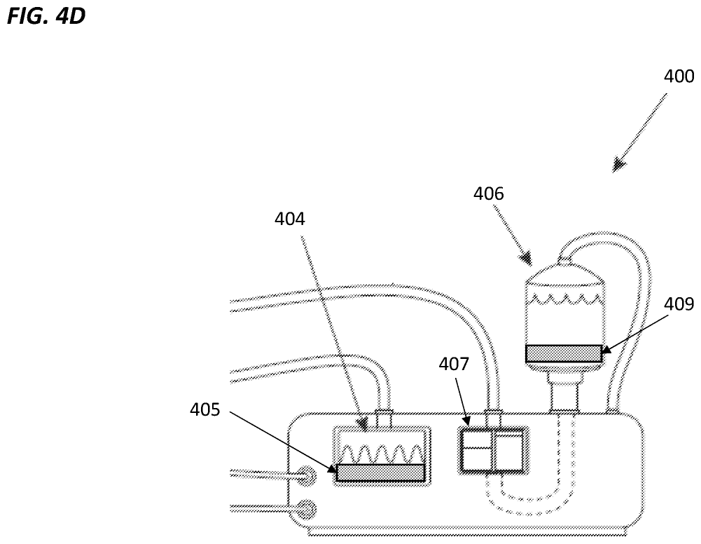

FIGS. 4A-4D illustrate various configurations of a thrombus removal system 400, including a thrombus removal device, 402, a vacuum source and cannister 404, a fluid source 406, and a pump 407. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 402 can include a funnel 408, a flexible shaft 410, a handle 412, and one or more controls 414 and 416. For example, in the embodiment shown in FIG. 4A, the device can include a finger switch or trigger 414 and a foot pedal or switch 416. These can be used to control aspiration and irrigation, respectively. Alternatively, as shown in the embodiment of FIG. 4B, the device can include only a foot switch 416, which can be used to control both functions, or in FIG. 4C, the device can include only an overpedal 416, also used to control both functions. It is also contemplated that an embodiment could include only a finger switch to control both aspiration and irrigation functions. As shown in FIG. 4A, the vacuum source and cannister 404 can be coupled to the aspiration lumen of the device with a vacuum line 418. Any clots or other debris removed from a patient during therapy can be received by, and stored in, the vacuum cannister 404 for later disposal. Similarly, the fluid source 406 (e.g., a saline bag) can be coupled to the fluid lumens of the device with a fluid line 420 for delivery of high-pressure fluid streams or jets at the base of funnel 408 to fragment thrombus material engaged by funnel 408, as described above.

During a thrombus removal procedure, referring to FIGS. 1, 1A, and 4A-4D, pressurized fluid is delivered from fluid source 406 and pump 407 through fluid line 420 and fluid lumen 45 to manifold 25 and ports 30. Simultaneously, vacuum is applied to aspiration lumen 55 by vacuum source and cannister 404. At the beginning of the procedure, the material flowing proximally through aspiration lumen 55 to the vacuum source and cannister is primarily fluid delivered through ports 30 combined with any blood that is able to pass around the engaged thrombus. As the fluid jets begin to break up the thrombus, thrombus material is pulled proximally through aspiration lumen 55 along with the injected fluid and any blood that can pass into the funnel around the thrombus. After the thrombus has been broken up, the remaining thrombus material moves proximally toward the vacuum source and cannister 604.

FIG. 4D is a close-up view of the console of the thrombus removal system, which can include the vacuum source and cannister 404 and the fluid source 406. In some embodiments, the cannister 404 and/or the fluid source 406 can include features designed and configured to assist in determining or estimating therapy progress, including determining or estimating the amount (e.g., volume, shape, or size) or percentage of clot removed (e.g., relative to a pre-treatment estimate of clot). Additionally, the cannister 404 and/or the fluid source 406 can include features designed and configured to assist in determining the amount of fluid (e.g., jets) delivered into the patient and/or the amount of blood removed or aspirated from the patient.

In FIG. 4D, cannister 404 can include sensor 405. In one embodiment, sensor 405 can comprise one or more weight scales configured to measure or sense the weight of fluids and biological materials inside the cannister. The weight scale(s) can be zeroed prior to therapy, and can provide real time weight measurement of the amount of fluid and/or biological materials removed or aspirated from the patient during a clot removal procedure.

In some embodiments, the cannister itself can include a drain or filter, with a drain size or filter configured to allow drainage of fluids from the cannister (such as blood and/or saline) while preventing clot material or other biological tissues from draining from the cannister. In this manner, the cannister and weight scale(s) are able to measure only the weight of the clot removed, and not blood and/or saline. The weight of the clot removed can also be used to estimate the volume of clot removed. Optionally, the blood and other fluids such as saline can be drained into a separate cannister (not shown), which can then be used to determine the amount of blood removed from the patient in addition to the amount of clot removed. In one implementation, fluid source 406 can also include sensor 409, which can be used to track the amount of fluid or saline delivered to the patient through jetting or irrigation. The sensor 409 can comprise, for example, additional weight scales, or optionally, any other sensor configured to measure the flow of fluid such as a flow sensor. The fluid delivered by the fluid source 406 can be measured by sensor 409 and subtracted from the fluid drained into the separate (not shown) cannister. Therefore, the amount of clot (e.g., weight and/or volume) can be determined with weight scales 407 in the cannister 404, and the amount of blood can be calculated in the separate container by subtracting the collected volume or weight of filtered fluids (e.g., blood and saline) from the amount of saline delivered as determined by sensor 409 of the fluid source.

In another embodiment, the sensor 405 on or in the cannister 404 can comprise a camera. In some embodiments, the camera can comprise a miniature or fiber optic camera. Additionally, multiple cameras in different imaging planes can be placed on or in the cannister. In some implementations, the camera can be configured to provide imaging of the cannister to provide a visual guide to the user regarding what is being aspirated from the patient. For example, the user can visualize the amount and/or size of clot being removed. The images from the camera can be displayed for the user, such as on a display that provides additional information about the status of the system, device, and procedure. The camera images can be displayed on the display in real-time during a procedure, to give the user or physician a live-look at the aspirated clot without having to look at the cannister itself. Alternatively, still or video images of the clot removed from the patient and captured by the cannister 404 can be used to determine or estimate the weight and/or volume of the clot removed. In some examples, one or more processors of the system can apply image processing techniques to the cannister images to estimate the size, weight, and/or volume of the clot. For example, imaging processing software can estimate or measure one or more dimensions of the removed clot(s) in one or more imaging planes. These measurements or estimates can then be used to estimate or determine the volume or weight of the removed clot(s).

In other embodiments, the sensor 405 on or in the cannister 404 can comprise other types of sensors, such as optical sensors, flow sensors, etc. Generally, the sensors can be used to monitor or characterize the amount and/or type of material or fluid that enters the cannister to provide the user with additional information regarding the status of the therapy.

In some embodiments, pre-operative medical imaging of the patient may be used for treatment planning, including estimating the size and/or volume of target clot(s) in the patient.

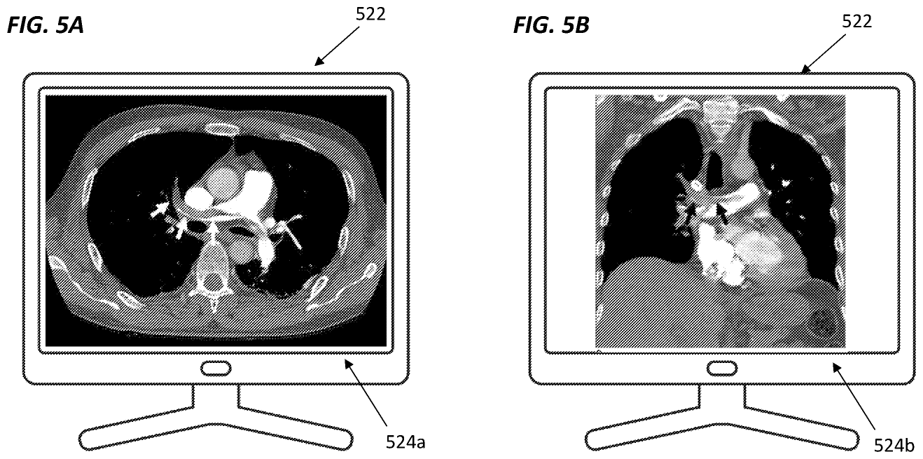

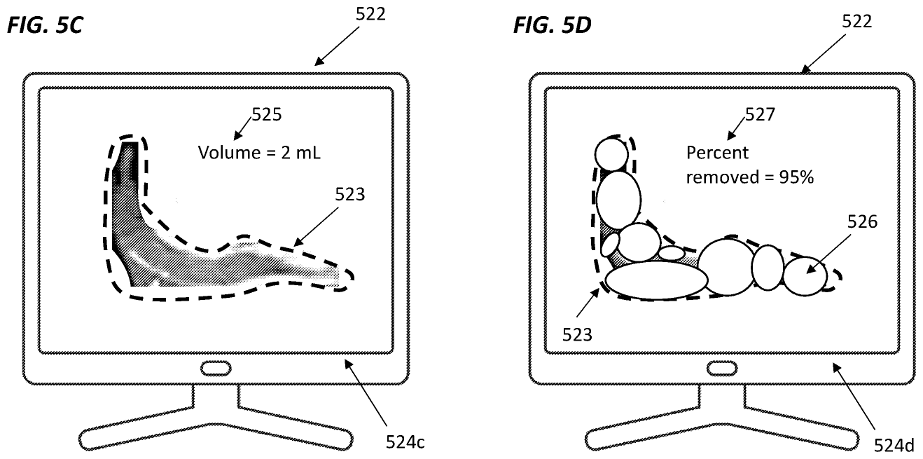

This imaging can include, but not be limited to, CT imaging, MRI, ultrasound, x-ray, and other suitable medical imaging systems. This imaging can be received by the thrombus removal system for treatment planning and determining or estimating treatment progress. Referring to FIGS. 5A-5B, a display 522 of the thrombus removal system can be configured to receive and/or display the medical imaging of the patient. In this example, an axial CT scan 524a of a patient's lungs is shown in FIG. 5A, and a coronal CT scan 524b of the patient's lungs is shown in FIG. 5B. Both scans show a large pulmonary embolism in the main pulmonary artery as indicated by the white and black arrows, respectively.

In one embodiment, the thrombus removal system can apply image processing or other techniques to estimate or calculate a parameter of the one or more clots, such as the size and/or volume of one or more clots or embolisms based on the medical imaging. In other aspects, the thrombus removal system can calculate or estimate a morphology of the one or more clots, such as the type of clot material including a hardness of the clot or a hardness of portions of the clot (e.g., soft clot, medium clot, hard clot, etc.). These estimates can be prior to therapy, while the clots are still inside the patient. For example, referring to FIGS. 5A-5B, the size/volume of the clot(s) can be estimated by evaluating one or more dimensions of an identified clot. In some examples, a plurality of slices in one or more imaging planes may be separately evaluated and then combined to improve the accuracy or quality of the estimated clot size/volume.

FIG. 5C illustrates an example of calculating or estimating a parameter of the clot(s), such as a clot volume/size estimation by the thrombus removal system based on the medical imaging. In this example, a pre-operative image 524c of the clot is displayed on display 522 of the thrombus removal system with information 525 pertaining to the estimated clot size and/or volume (e.g., an estimated volume of 2 mL is provided for this example clot). This estimated size and/or volume can be based on the image processing of the pre-operative medical images described above. In some embodiments, an outline 523 of the clot can be displayed in the shape of the pre-treatment clot from the medical imaging.

FIG. 5D provides an example of a treatment progress screen 524d on a display 522 of a thrombus removal system that can be presented to a user either during or after a thrombectomy procedure. In this example, estimates or calculations of the clot weight/size/volume removed from the patient (e.g., with sensors or cameras as described above) can be graphically overlaid on the pre-operative image or outline 523 of the clot from the medical imaging. In some embodiments, the estimates or calculations of the clot weight/size/volume come from the sensors 405 and/or 409 of the system described above in FIG. 4D. For example, the weight of the clot can be determined by the system and used to estimate the clot volume, or alternatively, image processing of images captured by camera(s) in the clot catcher or cannister can be used to estimate or determine the volume of clot removed. FIG. 5D shows individual broken-up or macerated clot pieces or chunks 526 overlaid on top of the image of the clot from the pre-operative imaging. As shown, the pieces or chunks of clot can be arranged and displayed so as to fill in the initial clot volume. In some examples, additional data 527 such as the percentage of clot removed or some other indicator of treatment progress can be displayed on the treatment progress screen 524d.

In some embodiments, information gleaned from the pre-operative imaging can be used to improve the accuracy of the clot size/volume estimates. For example, the location of the clot within the patient anatomy may provide information on the size of the clot. Specifically, pre-operative imaging can indicate if the clots are saddle pulmonary embolisms, lobar pulmonary embolisms, or distal pulmonary embolisms. The location of the clot provides information on the size of the clot, which the system can use in conjunction with other parameters (e.g., weight or measured/estimated dimensions) to provide accurate estimates as to size/volume.

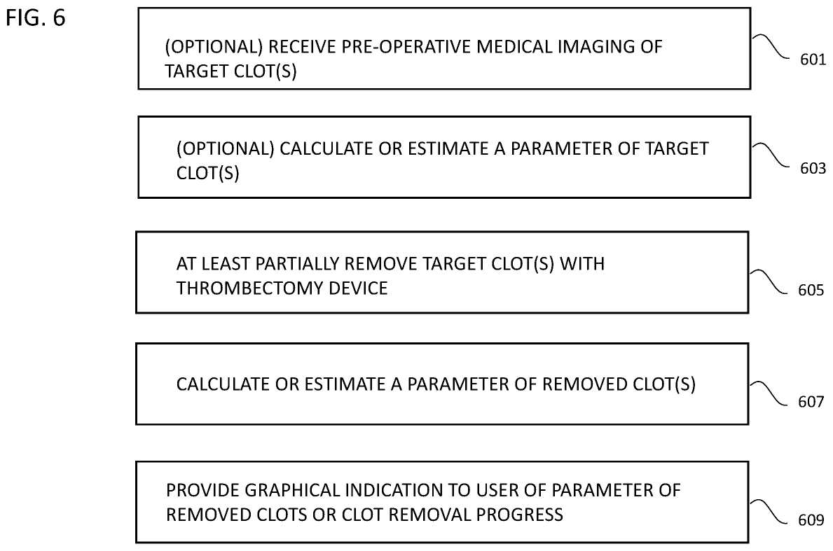

FIG. 6 is a flowchart describing a thrombectomy method. At step 601 of the flowchart, the method can include optionally receiving pre-operative medical imaging of one or more target clots to be removed. The pre-operative medical imaging can comprise, for example, CT or MRI imaging. In some aspects, more than one target clots are identified in the medical imaging.

At step 603, the method can optionally include calculating or estimating a parameter of the target clot(s). In some examples, the parameter is a size parameter, such as one or more dimensions of the target clot(s). In other examples, the parameter is a volume parameter, such as total volume of clot. In some aspects, the parameter is a weight parameter, such as a weight of the clot(s).

At step 605, the method can include removing or partially removing the one or more target clots with a thrombectomy device. In some aspects, removing the clots comprises aspirating the clots from the patient. Removing can further include breaking up or macerating the target clot with one or more jets or fluid streams of the thrombectomy device.

At step 607, the method can include calculating or estimating a parameter of the removed clot(s). The parameter can be the same parameter as from the optional step 603. For example, the calculated or estimated parameter can be a size dimension, a volume, or a weight of the removed clot. In some aspects, calculating or estimating the parameter of the removed clot can be achieved with one or more sensors of the thrombectomy system, including but not limited to weight sensors, flow sensors, cameras, optical sensors, or the like. In some aspects, blood and other fluids are filtered or separated from the removed clot to give a better estimate or calculation of the clot parameter.

At step 609, the method can include providing a visual indication to a user of the calculated clot parameter or of clot removal/treatment progress. In some aspects, the indication can include displaying the calculated parameter of the removed clot, such as displaying the size, volume, or weight of the removed clot(s). In other aspects, the method can include providing a visual indication of the removed clot. This can include reconstructing the broken up or macerated clots into a visual representation that is overlaid upon pre-operative imaging of the target clots.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method of estimating progress of a thrombectomy procedure, comprising:
performing pre-operative medical imaging of one or more clots in a patient;
determining a pre-operative parameter of the one or more clots based on the pre-operative medical imaging;
removing at least a portion of the one or more clots with a thrombectomy device;
determining a parameter of the removed portion of the one or more clots; and
displaying the parameter of the removed portion of the one or more clots to a user.

2. The method of claim 1, wherein the pre-operative medical imaging comprises CT imaging.

3. The method of claim 1, wherein the pre-operative parameter comprises a volume of the one or more clots.

4. The method of claim 1, wherein the pre-operative parameter comprises one or more dimensions of the one or more clots.

5. The method of claim 1, wherein the pre-operative parameter comprises a weight of the one or more clots.

6. The method of claim 1, wherein the pre-operative parameter comprises a morphology of the one or more clots.

7. The method of claim 1, wherein the parameter of the removed portion comprises a volume of the one or more clots removed.

8. The method of claim 1, wherein the parameter of the removed portion comprises one or more dimensions of the one or more clots removed.

9. The method of claim 1, wherein the parameter of the removed portion comprises a weight of the one or more clots removed.

10. The method of claim 1, wherein the parameter of the removed portion comprises a morphology of the one or more clots removed.

11. The method of claim 1, wherein determining the pre-operative parameter comprises performing image processing to estimate the parameter of the one or more clots.

12. The method of claim 1, wherein determining the parameter of the removed portion comprises sensing a weight of the one or more clots after removal.

13. The method of claim 12, wherein determining further comprises weighing the one or more clots in a clot capture container.

14. The method of claim 1, wherein determining the parameter of the removed portion comprises evaluating one or more images of the one or more clots after removal.

15. The method of claim 1, further comprising comparing the pre-operative parameter to the parameter of the removed portion of the one or more clots.

16. The method of claim 15, wherein further comprises displaying a percentage of the one or more clots removed.

17. The method of claim 1, further comprising overlaying a graphic of portions of the one or more clots removed on top of a graphic of the pre-operative medical imaging of the one or more clots.

18. The method of claim 1, further comprising filtering blood and/or saline from the one or more removed clots.

19. The method of claim 18, further comprising comparing a pre-operative weight of the one or more clots to a weight of the one or more clots removed.

20. The method of claim 18, further comprising comparing a pre-operative dimension of the one or more clots to a dimension of the one or more clots removed.

21. The method of claim 18, further comprising comparing a pre-operative volume of the one or more clots to a volume of the one or more clots removed.

22. The method of claim 1, wherein removing at least a portion of the one or more clots comprises:
directing one or more fluid streams towards the one or more clots to break up the one or more clots; and
aspirating the one or more clots with the thrombectomy device.

23. The method of claim 22, further comprising calculating a volume of saline delivered with the one or more fluid streams.

24. The method of claim 23, further comprising separating fluid from the removed portion of the one or more clots.

25. The method of claim 24, further comprising calculating a volume of blood removed from the patient by subtracting the volume of saline delivered from a volume of the separated fluid.

26-36. (canceled)
